(19) United States
(12) Patent Application Publication
(10) Pub. No.: US 2021/0121280 A1
(43) Pub. Date: Apr. 29, 2021

(54) Filter for Stent Retriever and Methods for Use Thereof

(71) Applicant: SANFORD HEALTH, Sioux Falls, SD (US)

(72) Inventors: Thomas HALDIS, Horace, ND (US); Alexander DROFA, West Fargo, ND (US)

(21) Appl. No.: 16/605,108

(22) PCT Filed: Apr. 16, 2018
(86) PCT No.: PCT/US2018/027751

§ 371 Date: Oct. 14, 2019

Related U.S. Application Data
(60) Provisional application No. 62/485,999, filed on Apr. 16, 2017.

(51) Int. Cl. A61F 2/01; A61F 2/82

(52) U.S. Cl. CPC A61F 2/01; A61F 2/82; A61F 2230/0069; A61F 2250/0039; A61F 2210/0014; A61F 2002/825; A61F 2230/0067

(57) ABSTRACT

Apparatus, systems and methods for use thereof are disclosed. An example apparatus includes a filter having an expandable frame and a semipermeable membrane coupled to the expandable frame. The expandable frame has a cone-shape in an expanded position. An apex of the expandable frame is arranged at the filter's first end. The expandable frame has a plurality of polygonal supports coupled together at the apex and that are radially biased outward such that in the expanded position the polygonal supports are spaced apart at a second end of the filter with the semipermeable membrane extending therebetween. The semipermeable membrane covers an area defined by the polygonal supports. A plurality of struts each have a first and second end, and the first end of each of the struts is coupled to a second end of the expandable frame. A stent is coupled to each of the second ends of the struts.

### RELATED APPLICATIONS

This application claims the benefit of priority to U.S. Provisional Application No. 62/485,999 entitled “Filter for Stent Retriever and Methods for Use Thereof,” filed on Apr. 16, 2017 that is hereby incorporated herein by reference in its entirety.

### BACKGROUND THE INVENTION

Cerebral blood flow is critical in human anatomy. If blood flow is blocked to the brain, the tissue that does not receive blood flow will become ischemic and begin to die. The result is either a deficit in cognition, function, or even death. The results are also irreversible if such an ischemia lasts too long. The period of time can vary from patient to patient, but typically if blood flow to the brain is not restored after approximately 24 hours, an ischemic stroke may occur. These ischemic strokes can happen in any of the cerebral arteries, but are most common in the middle cerebral artery. Traditionally, such middle cerebral artery acute ischemic strokes resulted in high morbidity and mortality.

Around 1995, the use of tissue plasminogen activator (tPA), a lytic agent, was introduced as the first treatment for ischemic stroke. The tPA brakes down unorganized acute clot. Unfortunately, less than 8% of patients are eligible. In 2015, a new approach was approved in the United States known as mechanical thrombectomy. In this approach, a stent or neurovascularization device, such as a stent retriever, is deployed within an occlusion, the stent struts spread into the occlusion, then the occlusion is drawn back into the catheter under suction and removed from the body. This mechanical thrombectomy approach has reduced the mortality rate by at least 50% for patients who are treated within the critical time window.

While outcomes are significantly improved, the mechanical thrombectomy procedure is still labor intensive. Specifically, the mechanical thrombectomy device is either introduced through a femoral or carotid access. If it is introduced from a femoral access, the operator needs to navigate the aortic anatomy with a special focus on the aortic arch. Aortic arches can be difficult to navigate, especially with type-three aortic arches. In addition, the tortuous nature of various arteries, such as the cerebral arteries, likewise provide navigational challenges to advance the stent retriever. Once the occlusion, such as an intracerebral clot, is reached the stent retriever is deployed across the occlusion for treatment. After the stent retriever has expanded and penetrated the occlusion, the stent retriever is then withdrawn back into an intermediate catheter that, in certain applications, typically resides in the distal internal carotid artery. As the stent retriever is withdrawn, suction is applied to the intermediate catheter and the stent retriever is captured in the intermediate catheter. The goal is to aspirate a majority of the clot under suction, but, invariably, a portion of the occlusion embolizes into a distal vessel, avoids capture by the stent retriever or is pulled into a neighboring branch vessel.

### SUMMARY OF THE INVENTION

The apparatus, systems and methods disclosed herein are contemplated for clot and emboli capture within vasculature that include, but are not limited to, the cerebral vessels, pulmonary arteries and the vena cava.

In particular, in a first aspect, the disclosure provides an apparatus that includes: (a) a filter having an expandable frame and a semipermeable membrane coupled to the expandable frame, the expandable frame has a cone-shape in an expanded position, an apex of the expandable frame is arranged at a first end of the filter, the expandable frame has a plurality of polygonal supports that are coupled together at the apex of the expandable frame and that are radially biased outward such that in the expanded position the plurality of polygonal supports are spaced apart at a second end of the filter with the semipermeable membrane extending therebetween, where the semipermeable membrane covers at least a portion of an area defined by the plurality of the polygonal supports, (b) a plurality of struts each having a first end and a second end, the first end of each of the plurality of struts coupled to a second end of the expandable frame; and (c) a stent coupled to each of the second ends of the plurality of struts.

In a second aspect, the disclosure provides an apparatus that includes: (a) a filter having an expandable frame, the expandable frame has a cone-shape in an expanded position, an apex of the expandable frame is arranged at a first end of the filter, the expandable frame has a plurality of polygonal supports that are coupled together at the apex of the expandable frame and that are radially biased outward such that in the expanded position an area defined by the plurality of the polygonal supports ranges from about 0.1 mm² to about 5.0 mm²; (b) a plurality of struts each having a first end and a second end, the first end of each of the plurality of struts coupled to a second end of the filter; (c) a cylindrical or cone-shaped cap coupled to the expandable frame at the apex; and (d) a stent coupled to each of the second ends of the plurality of struts.

In a third aspect, the disclosure provides a system that includes: (a) a first apparatus according to the first aspect; (b) a second apparatus according to the first aspect, where the stent of the second apparatus is coupled to the first end of the filter of the first apparatus; and (c) a third apparatus according to the first aspect, where the stent of the third apparatus is coupled to the first end of the filter of the second apparatus.

In a fourth aspect, the disclosure provides a system that includes: (a) a catheter having a first lumen and a second lumen, where the first lumen is in mechanical communication with a first opening defined at a first end of the catheter, where the second lumen is in mechanical communication with a second opening defined in a sidewall of the catheter at least 4 cm from the first end of the catheter; (b) a first apparatus according to the first or second aspect moveably disposed within the first lumen of the catheter; and (c) a second apparatus according to the first or second aspect moveably disposed within the second lumen of the catheter.

In a fifth aspect, the disclosure provides a system that includes: (a) a first catheter having a first lumen; (b) a second catheter having a second lumen that bifurcates into a third lumen and a fourth lumen at a first end of the second catheter, where the first lumen is in mechanical communication with a first opening defined at a first end of the first catheter, where the second catheter is moveably disposed within the first lumen of the first catheter such that at least a portion of the third lumen and the fourth lumen are configured to advance out of the first catheter and retract into the first catheter; (c) a first apparatus according to the first or second aspect moveably disposed within the third lumen of the second catheter, where the third lumen is in mechanical communication with a second opening defined at the first end of the second catheter; and (d) a second apparatus according to the first or second aspect moveably disposed within the fourth lumen of the second catheter, where the fourth lumen is in mechanical communication with a third opening defined at the first end of the second catheter.

In a sixth aspect, the disclosure provides a method that includes: (a) advancing an apparatus according to the first or second aspect across a clot such that the expandable frame of the filter is distal to the clot and the stent is disposed within the clot; (b) retracting a catheter, thereby permitting the expandable frame of the filter and the stent to expand such that the stent integrates into the clot; and (c) retracting the stent thereby causing any loose emboli to advance through the plurality of struts and into the expandable frame of the filter.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a side view of an apparatus, according to an example embodiment.

FIG. 2 illustrates a side view of the apparatus according to FIG. 1 disposed within a target lumen.

FIG. 3 illustrates a side view of the apparatus, according to an example embodiment.

FIG. 4 illustrates a side view of a system, according to an example embodiment.

FIG. 5 illustrates a side view of a system that includes a catheter, according to an example embodiment.

FIG. 6 illustrates a side view of a system that includes a catheter, according to another example embodiment.

### DETAILED DESCRIPTION OF THE INVENTION

Exemplary apparatus, systems and methods are described herein. The word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. Certain aspects of the disclosed apparatus, systems and methods can be arranged and combined in a wide variety of different configurations, all of which are contemplated herein.

Furthermore, the particular arrangements shown in the Figures should not be viewed as limiting. Other embodiments may include more or less of each element shown in a given Figure. Further, some of the illustrated elements may be combined or omitted. Yet further, an exemplary embodiment may include elements that are not illustrated in the Figures.

As used herein, with respect to measurements, “about” means+/−5%.

As used herein, a “catheter” is an apparatus that is connected to a deployment mechanism and is configured to house a medical device that can be delivered over a guidewire. The catheter may include a guidewire lumen for over-the-wire guidance and may be used for delivering the medical device, including a stent retriever, to a target lumen.

As used herein, “kite-shaped” refers to a quadrilateral with two distinct pairs of equal adjacent sides such that one diagonal is the perpendicular bisector of the other diagonal.

As used herein, “sinusoidal” refers to regular undulations of the outer wall of the stent retriever, when the stent retriever is in the expanded position.

As used herein, a “guidewire” is an elongated cable comprised of one or more biocompatible materials including metals and polymers. Guidewires may be used for selecting target lumens and guiding catheters to target deployment locations. Guidewires are typically defined as wires used independently of other devices that do not come as part of an assembly.

As used herein, a “stent” is a device that is advanced through emboli or a clot in the form of an occlusion and configured to expand and embed in the clot. Once embedded in the occlusion, the stent may then be retracted to restore blood flow and aid thrombectomy in acute embolic stroke.

As used herein, a “stent retriever” is one example of a self-expanding stent that has a thin rope of wire mesh in a compressed position and that is configured to expand into an out-pouching stent structure for integration into a clot in an expanded position.

As used herein, a “membrane” is thin pliable sheet of material.

As used herein, “lumen” refers to a passage within an arterial or tubular structure, such as the pulmonary arteries or a passage within the tubular housings or catheters through which the guidewire may be disposed.

As used herein, “first end” refers to a distal end of the device or component thereof, and “second end” refers to a proximal end of the device or component thereof.

As used herein, “distal” with respect to a portion of the apparatus means the end of the device (when in use) nearer the treatment zone (e.g., the middle cerebral artery or pulmonary artery) of the subject and the term “proximal” means the portion of the device (when in use) further away from the targeted lumen of the subject and nearer the access site and the operator.

As used herein, a system, apparatus, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware that enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

Exemplary devices and systems are described herein. It should be understood that the word “exemplary” is used herein to mean “serving as an example, instance, or illustration.” Any embodiment or feature described herein as “exemplary” is not necessarily to be construed as preferred or advantageous over other embodiments or features. The exemplary embodiments described herein are not meant to be limiting. It will be readily understood that certain aspects of the disclosed systems and methods can be arranged and combined in a wide variety of different configurations, all of which are contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

One benefit provided by the examples described herein is a filter apparatus, system and methods for use thereof arranged to capture embolic particles that are extruded through the struts of the stent retriever or that roll between the wall of the stent retriever and the wall of the lumen and ultimately off the distal end of the stent retriever and into the lumen. Another advantage of the filter apparatus, system and methods described herein is that the expandable frame and semipermeable membrane may be configured to conform to a lumen of a target vessel in an expanded position. For example, in one embodiment, the expandable frame is radially biased outward permitting the filter apparatus to conform to changes in the wall of the target lumen as the stent retriever is retracted to reduce or eliminate any gaps between the apparatus and the wall of the target lumen.

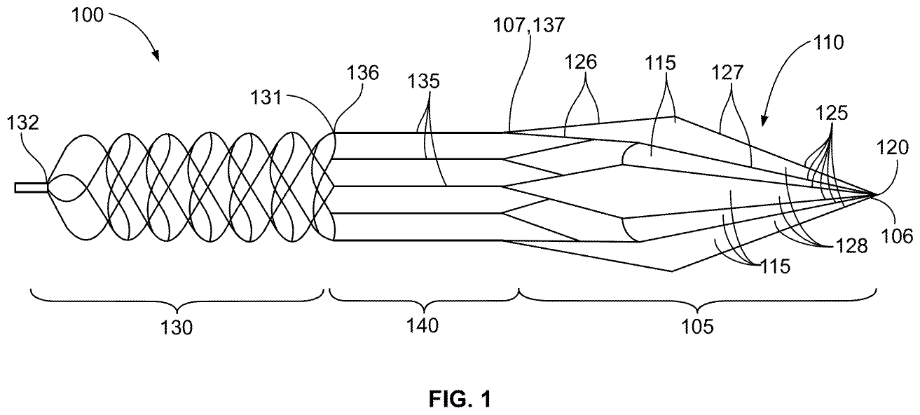

FIG. 1 depicts an apparatus 100 according to a first aspect that includes a filter 105 having an expandable frame 110 and a semipermeable membrane 115 coupled to the expandable frame 110. The expandable frame 110 has a cone-shape in an expanded position, shown in FIG. 1, and an apex 120 of the expandable frame 110 is arranged at a first end 106 of the filter 105. The expandable frame 110 has a plurality of polygonal supports 125 that are coupled together at the apex 120 of the expandable frame 110 and that are radially biased outward. In the expanded position of the expandable frame 110, the plurality of polygonal supports 125 are spaced apart at the second end 107 of the filter 105 with the semipermeable membrane 115 extending therebetween. The semipermeable membrane 115 also covers at least a portion of the area defined by the plurality of the polygonal supports 125.

In one example, the expandable frame 110 includes a material having shape memory, such as nitinol. The expandable frame 110 is self-expanding such that the filter 105 expands upon deployment, as a catheter is retracted relative to the apparatus 100. In another example, the expandable frame 110 has a length ranging from 1 cm to 300 cm. In a further example, the plurality of polygonal supports 125 of the expandable frame 110 are kite-shaped, as shown in FIG. 1, such that the polygonal supports 125 form a pair of short sides 126 and a pair of long sides 127. The pair of long sides 127 of each of the plurality of kite-shaped segments 128 are coupled together at the apex of the expandable frame 110.

Still further, the expandable frame 110 and semipermeable membrane 115 are optionally configured to conform to a lumen of a target vessel in an expanded condition. In one example, the semipermeable membrane 115 is pliable and the plurality of polygonal supports 125 are movable between the expanded position and a compressed position in which the plurality of polygonal supports 125 are compressed inward toward a central longitudinal axis of the expandable frame 110. In this example, the semipermeable membrane 115 may collapse or fold inward. This arrangement may beneficially permit the filter 105 to maintain apposition with the wall of a lumen in which the filter 105 is deployed by expanding and flexing to adjust to a wall of a target lumen or navigating from smaller vessels into larger vessels, for example. In a further optional example, the plurality of polygonal supports 125 include a material having shape-memory such that the plurality of polygonal supports 125 elongate in the compressed position and widen or spread apart in the expanded position. In one example, in the expanded position, a diameter of a second end 107 of the filter 105 ranges from about 3 mm to about 6 cm.

In various examples, the semipermeable membrane 115 has a plurality of openings ranging in size from 110 μm to 1000 μm. Further, the semipermeable membrane 110 is at least woven, porous, cross-hatched, or multi-layered. The semipermeable membrane 110 may be made of polymers or silk, for example. In addition, the semipermeable membrane 110 has a thickness ranging from about 0.001 mm to about 0.5 mm.

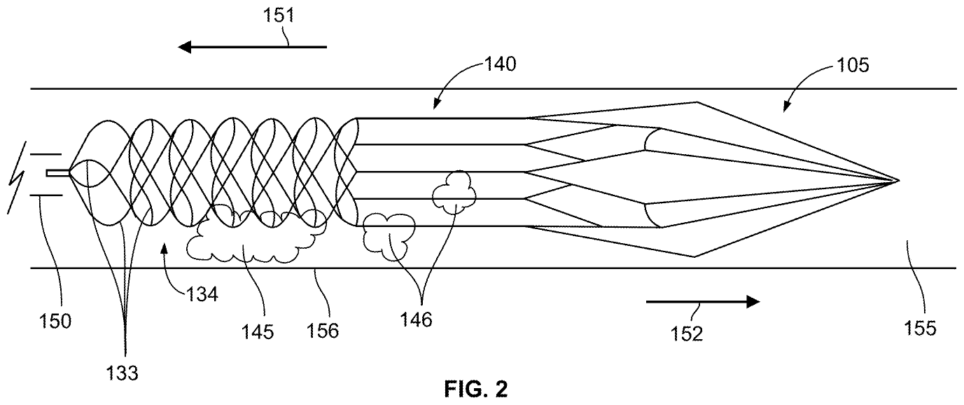

The filter 105 also includes a stent 130 having a first end 131 and a second end 132 and a plurality of struts 135 each having a first end 136 and a second end 137. The first end 136 of each of the plurality of struts 135 is coupled to a second end 107 of the filter 105, and the second end 137 of each of the plurality of struts 135 is coupled to a first end 131 of the first stent 130. In one example, a length of the plurality of struts 135 ranges from about 1 mm to about 10 cm. In addition, the plurality of struts 135 are configured to bend and adapt to tortuous anatomy and to have sufficient radial force to capture a clot and emboli. For example, the plurality of struts 135 are made of biocompatible shape-memory metals or alloys, such as nitinol, or shape-memory polymers with sufficient radial force. The plurality of struts 135 may have a hydrophilic coating or a heparin coating. The spacing of the plurality of struts 135 about the perimeter of the filter results in a clot 145 capture window 140 between the stent 130 and the filter 105. In one example implementation, shown in FIG. 2, once the stent 130 has been deployed and integrated into a clot 145, the stent 130 and filter 105 are retracted back into the catheter 150 in direction 151. Embolic particles 146 extruded through the struts 133 of the stent 130 or that roll between the wall 134 of the stent 130 and the wall 156 of the lumen 155 and ultimately off the distal second end 132 of the stent 130 may enter the clot capture window 140 between the plurality of struts 135 and then enter the second end 107 of the filter 105. The embolic particles 146 are subsequently retracted into the catheter 150 via the filter 105.

In one example, the stent 130 is a stent retriever. In another example, one or more of the expandable frame 110, the plurality of struts 135 and the stent 130 have a hydrophilic coating. The hydrophilic coating decreases resistance to advancement and retraction of the stent 130 within a target lumen. In a further example, the plurality of struts 135 are each coupled to one of the plurality of kite-shaped segments 128 at a location where the pair of short segments 126 is coupled together.

In alternative examples, a non-permeable membrane may be used in conjunction with the semipermeable membrane. For example, the non-permeable membrane may be disposed between the plurality of polygonal supports 125 and the semipermeable membrane 115 may cover all or a portion of the area defined by the plurality of polygonal supports 125 and vice versa. Such an arrangement disposes the non-permeable membrane between the semipermeable membrane 115 in an alternating fashion thereby permitting blood flow to be maintained through the semipermeable membrane 115 while increasing patency of the filter 105.

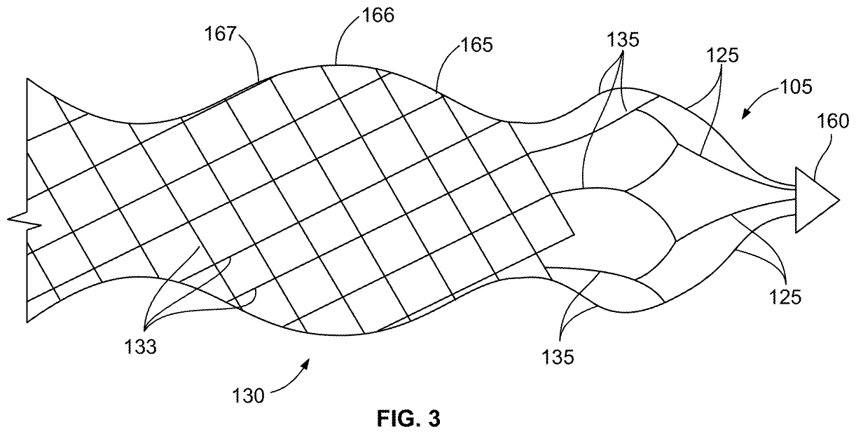

FIG. 3 depicts an apparatus 100 according to a third aspect that includes a filter 105 having an expandable frame 110. The expandable frame 110 has a cone-shape in an expanded position. An apex 120 of the expandable frame 110 is arranged at a first end 106 of the filter 105. The expandable frame 110 has a plurality of polygonal supports 125 that are coupled together at the apex 120 of the expandable frame 110 and that are radially biased outward such that in the expanded position an area defined by the plurality of the polygonal supports 125 ranges from about 0.1 mm² to about 5.0 mm. The area defined by the plurality of the polygonal supports 125 is sized to prevent passage of particles larger than 500 μm in stroke applications but could be larger in pulmonary embolism or deep venous thrombosis applications, while maintaining blood flow. The apparatus 100 includes a plurality of struts 135 each having a first end 136 and a second end 137. The first end 136 of each of the plurality of struts 135 is coupled to a second end 107 of the filter 105. The apparatus 100 in the second aspect also includes a cylindrical or cone-shaped cap 160 coupled to the expandable frame 110 at the apex 120. A stent 130 coupled to each of the second ends 137 of the plurality of struts 135. The cylindrical or cone-shaped cap 160 facilitates clot capture. In an alternative embodiment, the cap 160 may have the form of a welded junction between the plurality of polygonal supports 125 at the apex 120 of the expandable frame 110.

In one example, an outer wall 134 of the stent 130 is sinusoidal having a plurality of undulations 165. The plurality of undulations 165 each have a higher pick density on a distal side 166 and a lower pick density on a proximal side 167. The higher pick density results in smaller cell size between the struts of the stent relative to the cell size of the lower pick density. This arrangement permits emboli to pass into the stent 130 on the proximal side 167 and to prevent or limit passage of the emboli out of the distal side 166 of the stent 130. In various other embodiments, the stent 130 may have an outer wall 134 that is cylindrical or bows outwardly in the mid-point, similar to a barrel.

In another example, a diameter of the stent 130 in the expanded position tapers at the first end 131. The tapered portion of the stent 130 may extend along 20-100% of the total length of the stent 130 in the expanded position. The taper of the stent 130 may advantageously permit the first end 131 of the stent 130 to extend into smaller vasculature.

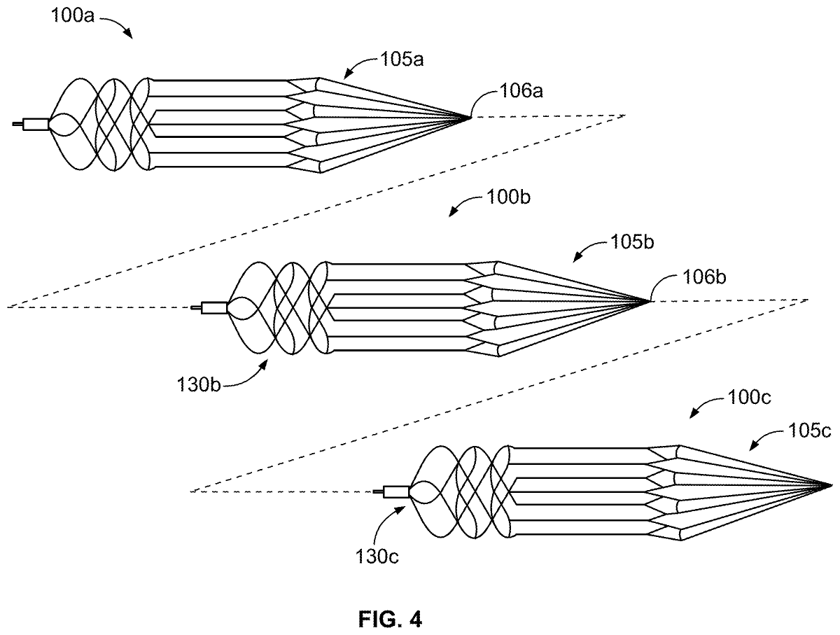

FIG. 4 depicts a system according to a third aspect that includes at least three apparatus 100a, 100b, 100c according to the first aspect described above, where the at least three apparatus 100a, b, c are arranged in series. In one example implementation, a first apparatus 100a according to the first or second aspect is provided and a second apparatus 100b according to the first or second aspect has a stent 130b coupled to the first end 106a of the filter 105a of the first apparatus 100a. And a third apparatus 100c according to the first aspect has a stent 130c coupled to the first end 106b of the filter 105b of the second apparatus 100b. This arrangement may advantageously increase the amount of emboli 146 captured by the filters 105a, b, c of the first, second and third apparatus 100a, b, c. In an optional example, shown in FIG. 3, the plurality of supports of the third apparatus 100c is greater than the plurality of supports of the second apparatus 100b and the plurality of supports of second apparatus of the second apparatus 100b is greater than the plurality of supports of the first apparatus 100a.

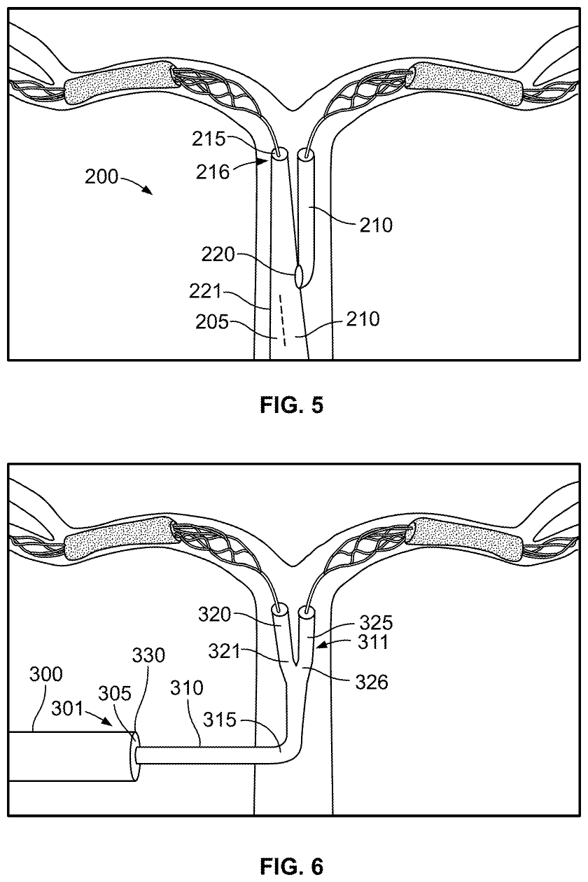

In a fourth aspect, shown in FIG. 5, systems are described that include a catheter to treat pulmonary embolism. In one example implementation of a system shown in FIG. 5, the system includes a catheter 200 that has a first lumen 205 and a second lumen 210. The first lumen 205 is in mechanical communication with a first opening 215 defined at a first end 216 of the catheter. The second lumen 210 is in mechanical communication with a second opening 220 defined in a sidewall 221 of the catheter 200 at least 4 cm from the first end of the catheter. A first apparatus 100 according to the first or second aspect is moveably disposed within the first lumen of the catheter. A second apparatus 100 according to the first or second aspect is moveably disposed within the second lumen 220 of the catheter 200. This arrangement may beneficially permit the first apparatus to be advanced into the first branch of the pulmonary arteries and the second apparatus to be advanced into the second branch of the pulmonary arteries. This has the advantage of deploying a single catheter thereby decreasing surgical time and complications. This arrangement may further permit increased integration of the stent into a saddle pulmonary embolism (PE) in the form of a large pulmonary thrombo-embolism that straddles the main pulmonary arterial trunk at the bifurcation and increase the effect of stent retraction of the pulmonary embolism clot.

In a fifth aspect shown in FIG. 6, the system includes a first catheter 300 having a first lumen 305 and a second catheter 310 having a second lumen 315 that bifurcates into a third lumen 320 and a fourth lumen 325 at a first end 311 of the second catheter. The first lumen 305 is in mechanical communication with a first opening 330 defined at a first end 301 of the first catheter 300. The second catheter 310 is moveably disposed within the first lumen 305 of the first catheter 300 such that at least a portion of the third lumen 320 and the fourth lumen 325 are configured to advance out of the first catheter 300 and retract into the first catheter 300. A first apparatus according to the first or second aspect is moveably disposed within the third lumen 320 of the second catheter 310. The third lumen 320 is in mechanical communication with a second opening 321 defined at the first end 311 of the second catheter 310. A second apparatus according to the first or second aspect is moveably disposed within the fourth lumen 325 of the second catheter 310. The fourth lumen 325 is in mechanical communication with a third opening 326 defined at a first end 311 of the second catheter. This arrangement may beneficially permit the first apparatus to be advanced from the third lumen 320 in to the first branch of the pulmonary arteries and permit the second apparatus to be advance from the fourth lumen 325 into the second branch of the pulmonary arteries. This has the advantage of deploying the first catheter 300 through tortuous anatomy and then advancing the second catheter 310 a short distance to the branch in the pulmonary arteries again decreasing surgical time and complications. This arrangement may further permit increased integration of the stent into a saddle pulmonary embolism (PE) in the form of a large pulmonary thrombo-embolism that straddles the main pulmonary arterial trunk at the bifurcation and increase the effect of stent retraction of the pulmonary embolism clot.

In a sixth aspect, methods are provided. In one example implementation, the method includes advancing an apparatus 100 according to the first or second aspect across a clot 145 such that the expandable frame 110 of the filter 105 is distal to the clot 145 and the stent 130 is disposed within the clot 145. Then, a catheter 150 is retracted, thereby permitting the expandable frame 110 of the filter 105 and the stent 130 to expand such that the stent 130 integrates into the clot 145. Next, the stent 130 is retracted thereby causing any loose emboli 146 to advance through the plurality of struts 135 and into the expandable frame 110 of the filter 105.

1. An apparatus, comprising:
a filter having an expandable frame and a semipermeable membrane coupled to the expandable frame, the expandable frame has a cone-shape in an expanded position, an apex of the expandable frame is arranged at a first end of the filter, the expandable frame has a plurality of polygonal supports that are coupled together at the apex of the expandable frame and that are radially biased outward such that in the expanded position the plurality of polygonal supports are spaced apart at a second end of the filter with the semipermeable membrane extending therebetween, wherein the semipermeable membrane covers at least a portion of an area defined by the plurality of the polygonal supports;
a plurality of struts each having a first end and a second end, the first end of each of the plurality of struts coupled to a second end of the filter; and
a stent coupled to each of the second ends of the plurality of struts.

2.-18. (canceled)

19. An apparatus, comprising:
a filter having an expandable frame and a semipermeable membrane coupled to the expandable frame, the expandable frame has a cone-shape in an expanded position, an apex of the expandable frame is arranged at a first end of the filter, the expandable frame has a plurality of polygonal supports that are coupled together at the apex of the expandable frame and that are radially biased outward such that in the expanded position an area defined by the plurality of the polygonal supports ranges from about 0.1 mm² to about 5.0 mm², wherein the semipermeable membrane covers at least a portion of an area defined by the plurality of the polygonal supports;
a plurality of struts each having a first end and a second end, the first end of each of the plurality of struts coupled to a second end of the filter;
a cylindrical or cone-shaped cap coupled to the expandable frame at the apex; and
a stent coupled to each of the second ends of the plurality of struts.

20. The apparatus of claim 19, wherein the plurality of polygonal supports are movable between the expanded position and a compressed position in which the plurality of polygonal supports are compressed inward toward a longitudinal axis of the expandable frame.

21. The apparatus of claim 19, wherein the plurality of polygonal supports are comprised of material having shape-memory such that the plurality of polygonal supports elongate in the compressed position and widen in the expanded position.

22. The apparatus of claim 19, wherein the plurality of polygonal supports of the expandable frame are kite-shaped having a pair of short sides and a pair of long sides, the pair of long sides of each of the plurality of kite-shaped segments coupled together at the apex of the expandable frame.

23. The apparatus of claim 22, wherein the plurality of struts are each coupled to one of the plurality of kite-shaped segments at a location where the pair of short segments is coupled together.

24. The apparatus of claim 19, wherein a length of the plurality of struts ranges from 1 mm to 10 cm.

25. The apparatus of claim 19, wherein the expandable frame is comprised of a material having shape memory and the expandable frame is self-expanding.

26. The apparatus of claim 19, wherein the expandable frame is comprised of nitinol and has a length ranging from 1 cm to 300 cm.

27. The apparatus of claim 19, wherein one or more of the expandable frame, the plurality of struts and the stent have a hydrophilic coating.

28. The apparatus of claim 19, wherein the expandable frame is configured to conform to a lumen of a target vessel in an expanded condition.

29. The apparatus of claim 19, wherein the stent is a stent retriever.

30. The apparatus of claim 19, wherein an outer wall of the stent is sinusoidal having a plurality of undulations, wherein the plurality of undulations each have a higher pick density on a proximal side and a lower pick density on a distal side.

31. The apparatus of claim 19, wherein a diameter of the stent in the expanded position tapers at the first end.

32. The apparatus of claim 19, wherein the plurality of struts are comprised of nitinol.

33. The apparatus of claim 19, wherein a diameter of a second end of the filter ranges from about 3 mm to about 6 cm in the expanded position.

34. A system, comprising:
a first apparatus according to claim 19;
a second apparatus according to claim 19, wherein the stent of the second apparatus is coupled to the first end of the filter of the first apparatus; and
a third apparatus according to claim 19, wherein the stent of the third apparatus is coupled to the first end of the filter of the second apparatus.

35. A system, comprising:
a catheter having a first lumen and a second lumen, wherein the first lumen is in mechanical communication with a first opening defined at a first end of the catheter, wherein the second lumen is in mechanical communication with a second opening defined in a sidewall of the catheter at least 4 cm from the first end of the catheter;
a first apparatus according to claim 19 moveably disposed within the first lumen of the catheter; and
a second apparatus according to claim 19 moveably disposed within the second lumen of the catheter.

36. A system, comprising:
a first catheter having a first lumen;
a second catheter having a second lumen that bifurcates into a third lumen and a fourth lumen at a first end of the second catheter, wherein the first lumen is in mechanical communication with a first opening defined at a first end of the first catheter, wherein the second catheter is moveably disposed within the first lumen of the first catheter such that at least a portion of the third lumen and the fourth lumen are configured to advance out of the first catheter and retract into the first catheter;
a first apparatus according to claim 19 moveably disposed within the third lumen of the second catheter, wherein the third lumen is in mechanical communication with a second opening defined at the first end of the second catheter; and
a second apparatus according to claim 19 moveably disposed within the fourth lumen of the second catheter, wherein the fourth lumen is in mechanical communication with a third opening defined at the first end of the second catheter.

37. A method, comprising:
advancing an apparatus according to claim 19 across a clot such that the expandable frame of the filter is distal to the clot and the stent is disposed within the clot;
retracting a catheter, thereby permitting the expandable frame of the filter and the stent to expand such that the stent integrates into the clot; and
retracting the stent thereby causing any loose emboli to advance through the plurality of struts and into the expandable frame of the filter.

38. The method of claim 37, further comprising:
the expandable frame of the filter conforming to a target lumen in which the filter resides.
